(19) United States
(12) Patent Application Publication
(10) Pub. No.: US 2004/0158321 A1
(43) Pub. Date: Aug. 12, 2004

(54) Method of implanting a mitral valve therapy device

(75) Inventors: David G. Reuter, Bothell, WA (US); Mark L. Mathis, Kirkland, WA (US)

Correspondence Address: GRAYBEAL JACKSON HALEY LLP, Suite. 350, 155-108th Avenue N.E., Bellevue, WA, 98004-5973, US

(73) Assignee: Cardiac Dimensions, Inc.

(21) Appl. No.: 10/366,585

(22) Filed: Feb. 12, 2003

(51) Int. Cl. A61F 2/24

(52) U.S. Cl. 623/2.36; 623/904

(57) ABSTRACT

There is disclosed a method of implanting a mitral valve therapy device in a patient's coronary sinus adjacent the patient's mitral valve annulus. The method includes the steps of positioning the mitral valve therapy device within the coronary sinus of the patient adjacent to the mitral valve annulus, evaluating effectiveness of the device, and assessing arterial perfusion of the heart.

### FIELD OF THE INVENTION

The present invention generally relates to methods of affecting the geometry of a heart via access through the cardiac venous system. The present invention also generally relates to a method of affecting the mitral valve annulus of a heart. The present invention more particularly relates to a method of implanting a mitral valve therapy device wherein the device is deployed and anchored in the coronary sinus of a heart adjacent the mitral valve annulus to reshape the mitral valve annulus.

### BACKGROUND OF THE INVENTION

The human heart generally includes four valves. Of these valves, a most critical one is known as the mitral valve. The mitral valve is located in the left atrial ventricular opening between the left atrium and left ventricle. The mitral valve is intended to prevent regurgitation of blood from the left ventricle into the left atrium when the left ventricle contracts. In preventing blood regurgitation the mitral valve must be able to withstand considerable back pressure as the left ventricle contracts.

The valve leaflets of the mitral valve are anchored to muscular wall of the heart by delicate but strong fibrous cords in order to support the leaflets during left ventricular contraction. In a healthy mitral valve, the geometry of the mitral valve ensures that the leaflets overlie each other to preclude regurgitation of the blood during left ventricular contraction.

The normal functioning of the mitral valve in preventing regurgitation can be impaired by dilated cardiomyopathy caused by disease or certain natural defects. For example, certain diseases may cause dilation of the mitral valve annulus. This can result in deformation of the mitral valve geometry to cause ineffective closure of the mitral valve during left ventricular contraction. Such ineffective closure results in leakage through the mitral valve and regurgitation. Diseases such as bacterial inflammations of the heart or heart failure can cause the aforementioned distortion or dilation of the mitral valve annulus. Needless to say, mitral valve regurgitation must not go uncorrected.

One method of repairing a mitral valve having impaired function is to completely replace the valve. This method has been found to be particularly suitable for replacing a mitral valve when one of the leaflets has been severely damaged or deformed. While the replacement of the entire valve eliminates the immediate problem associated with a dilated mitral valve annulus, presently available prosthetic heart valves do not possess the same durability as natural heart valves.

Various other surgical procedures have been developed to correct the deformation of the mitral valve annulus and thus retain the intact natural heart valve function. These surgical techniques involve repairing the shape of the dilated or deformed valve annulus. Such techniques, generally known as annuloplasty, require surgically restricting the valve annulus to minimize dilation. Here, a prosthesis is typically sutured about the base of the valve leaflets to reshape the valve annulus and restrict the movement of the valve annulus during the opening and closing of the mitral valve.

Many different types of prostheses have been developed for use in such surgery. In general, prostheses are annular or partially annular shaped members which fit about the base of the valve annulus. The annular or partially annular shaped members may be formed from a rigid material, such as a metal, or from a flexible material.

While the prior art methods mentioned above have been able to achieve some success in treating mitral regurgitation, they have not been without problems and potential adverse consequences. For example, these procedures require open heart surgery. Such procedures are expensive, are extremely invasive requiring considerable recovery time, and pose the concomitant mortality risks associated with such procedures. Moreover, such open heart procedures are particularly stressful on patients with a compromised cardiac condition. Given these factors, such procedures are often reserved as a last resort and hence are employed late in the mitral regurgitation progression. Further, the effectiveness of such procedures is difficult to assess during the procedure and may not be known until a much later time. Hence, the ability to make adjustments to or changes in the prostheses to obtain optimum effectiveness is extremely limited. Later corrections, if made at all, require still another open heart surgery.

An improved therapy to treat mitral regurgitation without resorting to open heart surgery has recently been proposed. This is rendered possible by the realization that the coronary sinus of a heart is near to and at least partially encircles the mitral valve annulus and then extends into a venous system including the great cardiac vein. As used herein, the term “coronary sinus” is meant to refer to not only the coronary sinus itself but in addition, the venous system associated with the coronary sinus including the great cardiac vein. The therapy contemplates the use of a device introduced into the coronary sinus to reshape and advantageously affect the geometry of the mitral valve annulus.

One such device includes an elongated flexible member having a cross sectional dimension for being received within the coronary sinus of the heart. The device includes an anchor at each of its ends. When placed in the coronary sinus, anchored and drawn taught, the device exerts an inward pressure on the mitral valve. The inward pressure increases the radius of curvature of the mitral valve annulus, or at least a portion of it, to promote effective valve sealing action and eliminate mitral regurgitation.

Such devices may be implanted in the coronary sinus using only percutaneous techniques similar to the techniques used to implant cardiac leads such as pacemaker leads. One prior proposed system for implanting the device includes an elongated introducer configured for being releasably coupled to the device. The introducer is preferably flexible to permit it to advance the device into the heart and into the coronary sinus through the coronary sinus ostium. To promote guidance, an elongated sheath is first advanced into the coronary sinus. Then, the device and introducer are moved through a lumen of the sheath until the device is in position within the coronary sinus. Because the device is formed of flexible material, it conforms to the curvatures of the lumen as it is advanced through the sheath. The sheath is then partially retracted. The distal end of the device is then anchored. Then, the sheath is retracted proximally. The introducer is then drawn proximally to place the device in tension. The sheath is then retracted further proximally past the proximal end of the device, where upon the proximal anchor is set. The procedure is then completed by the release of the introducer from the device and retraction of the introducer and sheath. As a result, the device is left within the coronary sinus to exert the inward pressure on the mitral valve annulus.

The foregoing therapy has many advantages over the traditional open heart surgery approach. Since the therapy may be employed in a comparatively noninvasive procedure, mitral valve regurgitation may be treated at an early stage in the mitral regurgitation progression. Further, the therapy may be employed with relative ease by any minimally invasive cardiologist. Still further, since the heart remains completely intact throughout the procedure, the effectiveness of the procedure in reducing mitral valve regurgitation may be readily determined, such as by echocardiography or fluoroscopy. Moreover, should adjustments be deemed desirable, such adjustments may be made during the procedure and before the patient is sent to recovery.

Unfortunately, the human anatomy does impose some obstacles to this recently proposed procedure for treating mitral regurgitation. More specifically, the coronary sinus/great cardiac vein runs in the atrioventricular groove between the left atrium and left ventricle. The left circumflex artery originates from the left main coronary artery and courses within the atrioventricular groove. One to three large obtuse marginal branches extend from the left circumflex artery as it passes down the atrioventricular groove. These principal branches supply blood to (perfuse) the lateral free wall of the left ventricle. In approximately 15% of the population, the left circumflex artery is a dominant source of blood to the left posterior descending artery for perfusing and supporting the viability of the left ventricle. When the circumflex artery is superior to the coronary sinus, the obtuse marginal branches extending towards the ventricular wall may run either underneath the coronary sinus or above the coronary sinus. Hence, when placing a mitral valve therapy device in the coronary sinus/great cardiac vein of a patient, great care must be taken to prevent occlusion of this coronary artery system.

### SUMMARY OF THE INVENTION

Even when great care is taken to avoid occlusion of the coronary arteries during placement of a prosthetic device in the cardiac venous system, arterial perfusion of the heart may be unacceptably reduced by the device. The present invention therefore provides a method of optimizing patient outcome while performing a procedure in the venous system of a patient's heart. The method includes the steps of performing a procedure in the venous system of the patient's heart, evaluating effectiveness of the procedure, and assessing arterial perfusion of the heart.

The method may further include the step of performing a further procedure in the venous system of the patient's heart after the evaluating step. The performing step may include positioning a mitral valve therapy device within the coronary sinus adjacent to the mitral valve annulus of the patient's heart. The method may include the further step of repositioning the device or removing the device after the evaluating step.

The present invention further provides a method of implanting a mitral valve therapy device in a patient's coronary sinus adjacent the patient's mitral valve annulus. The method includes the steps of positioning the mitral valve therapy device within the coronary sinus of the patient adjacent to the mitral valve annulus of the patient, evaluating effectiveness of the device, and assessing arterial perfusion of the heart.

The method may include the further step of adjusting the position of the device or removing the device after the assessing step. The device may include a distal anchor and a proximal anchor, the positioning step may include deploying the distal anchor within the coronary sinus, and the evaluating step may include pulling proximally on the device. The assessing step is preferably performed as the device is pulled proximally. The proximal anchor may then be deployed while pulling proximally on the device. The effectiveness of the device may be confirmed after deploying the proximal anchor. Arterial perfusion of the heart may also be assessed after deploying the proximal anchor. The method may further include the step of recapturing the proximal anchor or removing the device after the deploying and assessing steps.

The assessing step may include performing coronary angiography, intravascular ultrasound, fractional flow reserve analysis, an echocardiography, detecting for myocardial ischemia, or detecting a chemical marker of ischemia. The step of detecting for myocardial ischemia may include taking an electrocardiogram.

The method may further include the step of determining anatomical features of the coronary sinus adjacent to the mitral valve annulus. The determined anatomical features may include one of shape, diameter, and length of the coronary sinus. The method may further include the steps of providing a plurality of mitral valve therapy devices, each device corresponding to a respective different set of the anatomical features and selecting one of the plurality of mitral valve therapy devices after determining the anatomical features of the coronary sinus.

The invention further provides a method of implanting a mitral valve therapy device in a patient's coronary sinus adjacent the patient's mitral valve annulus. The device may include a distal anchor and a proximal anchor. The method includes the steps of positioning the mitral valve therapy device within the coronary sinus adjacent to the mitral valve annulus, deploying the distal anchor, evaluating effectiveness of the device, performing an arterial perfusion assessment of the heart, deploying the proximal anchor, and performing a second arterial perfusion assessment of the heart.

The method may further include the step of recapturing the distal anchor after the step of performing an arterial perfusion assessment of the heart. The method may further include the step of recapturing the proximal anchor after the step of performing a second arterial perfusion assessment of the heart.

### BRIEF DESCRIPTION OF THE DRAWINGS

The features of the present invention which are believed to be novel are set forth with particularity in the appended claims. The invention, together with further aspects and advantages thereof, may best be understood by making reference to the following description taken in conjunction with the accompanying drawings, in the several figures of which like reference numerals identify identical elements, and wherein:

FIG. 1 is a superior view of a human heart with the atria removed;

FIG. 2 is a superior view of a human heart similar to FIG. 1 illustrating a mitral valve therapy device deployed in the coronary sinus by a method embodying the present invention;

FIG. 3 is a superior view similar to FIG. 1 with portions cut away illustrating a step in determining anatomical features of the coronary sinus;

FIG. 4 is a superior view similar to FIG. 1 illustrating a further step taken to determine anatomical features;

FIG. 5 is a view similar to FIG. 1 illustrating a step in determining length dimensions of the coronary sinus/great cardiac vein;

FIG. 6 is a superior view similar to FIG. 1 illustrating the device being positioned adjacent the mitral valve annulus within the coronary sinus; and

FIG. 7 is a superior view similar to FIG. 1 illustrating the deployment of a distal anchor of the device.

### DETAILED DESCRIPTION OF THE INVENTION

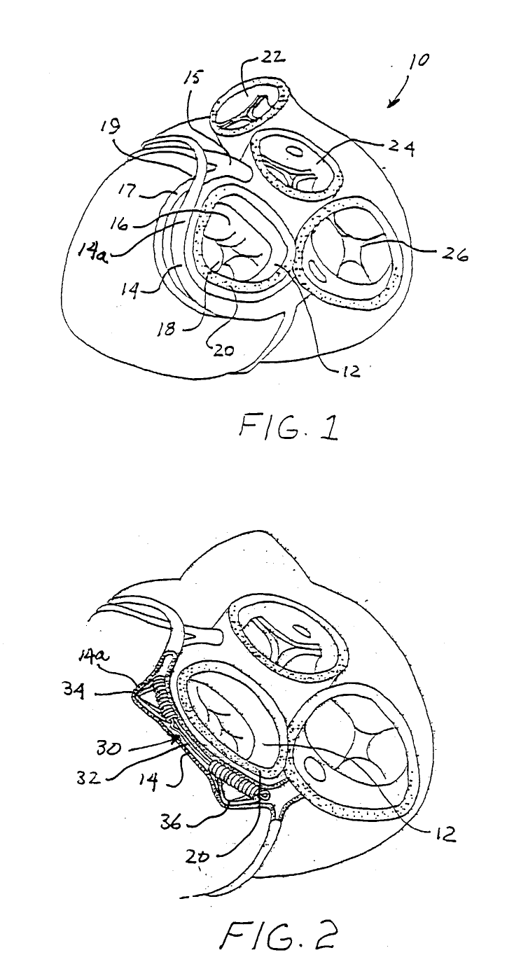

While the invention pertains generally to implanting prosthetic devices in the cardiac venous system, the invention can be illustrated by reference to a procedure performed in and around the coronary sinus and great cardiac vein. Referring now to FIG. 1, it is a superior view of a human heart 10 with the atria removed to expose the mitral valve 12, the coronary sinus 14, the coronary artery 15, and the circumflex artery 17 of the heart 10 to lend a better understanding of the present invention. Also generally shown in FIG. 1 are the pulmonary valve 22, the aortic valve 24, and the tricuspid valve 26 of the heart 10. The coronary sinus 14 as previously defined herein includes the great cardiac vein. As is well known, the coronary sinus becomes the great cardiac vein at some point. Hence, for purposes of describing the implant method of the present invention, the great cardiac vein or great vein 14a will be referred to as it particularly pertains to the distal end of the device to be implanted.

The mitral valve 12 includes an anterior leaflet 16, a posterior leaflet 18 and an annulus 20. The annulus encircles the leaflets 16 and 18 and maintains their spacing to provide a complete closure during a left ventricular contraction. As is well known, the coronary sinus 14 (and great vein 14a) partially encircles the mitral valve 12 adjacent to the mitral valve annulus 20. As is also known, the coronary sinus (and great vein) is part of the venus system of the heart and extends along the AV groove between the left atrium and the left ventricle. This places the coronary sinus essentially within the same plane as the mitral valve annulus making the coronary sinus available for placement of the mitral valve therapy device of the present invention therein.

Of particular importance is the physiological relationship of the coronary sinus 14 and the circumflex artery 17. The circumflex artery 17 branches from the coronary artery 15 and supplies blood flow to critical tissue of the heart 10. The circumflex artery passes beneath the coronary sinus 14 such as at crossover point 19 as shown in FIG. 1. It is one aspect of the present invention to avoid constriction of blood flow through the circumflex artery 17 and its branches when a mitral valve therapy device is deployed in the coronary sinus 14 (great vein 14a).

FIG. 2 shows a mitral valve therapy device 30 deployed in the coronary sinus 14 of the heart 10 adjacent the mitral valve annulus 20 for affecting the geometry of the mitral valve annulus. The device 30 takes the form of an elongated body 32 which includes a distal anchor 34 and a proximal anchor 36.

The anchors 34 and 36 are shown in FIG. 2 in their deployed configuration. A more complete description of the anchors 34 and 36 and their deployment may be had in copending application Ser. No. 10/142,637, filed May 8, 2002 for BODY LUMEN DEVICE ANCHOR, DEVICE AND ASSEMBLY which is assigned to the assignee of the present invention and hereby incorporated herein by reference. As will be seen hereinafter, in deploying the device 30 in the coronary sinus 14, the distal anchor 34 is first deployed in the great vein 14a to anchor the distal end of the device 30. In the anchoring process, the anchor 34 is expanded outwardly to anchor the device in the great vein 14a against both bi-directional longitudinal and rotational movement. This allows the device 30 to be tightened within the coronary sinus by pulling of the device's proximal end. Then, the proximal anchor 36 is deployed. The device 30, which may be formed from Nitinol or stainless steel, for example, now exerts an inward pressure on the mitral valve annulus 20 to advantageously affect its geometry.

The implant of the device 30 is initiated with an assessment of the degree of mitral regurgitation being suffered by the patient. This is accomplished by performing an echocardiogram to document the degree of mitral regurgitation. The echocardiogram may be either a transthoracic echocardiogram or a transesophageal echocardiogram.

Once the degree of mitral regurgitation is assessed, the coronary sinus 14 as illustrated in FIG. 3 is cannulated. The coronary sinus 14 is cannulated with a catheter 40 which is inserted through the ostium 13 of the coronary sinus 14 and into the coronary sinus. The distal end 42 of the catheter 40 is positioned in the proximal coronary sinus. With the catheter 40 thus positioned, a venogram of the coronary sinus is performed to define the coronary sinus anatomy and diameter. The venogram may be performed in a manner well known in the art wherein a contrast material 44 is injected into the coronary sinus for viewing under fluoroscopy.

After the venogram of the coronary sinus, the circumflex artery is cannulated in a manner well known in the art. An angiogram, also as known in the art, is then performed to define the baseline circumflex/obtuse marginal anatomies.

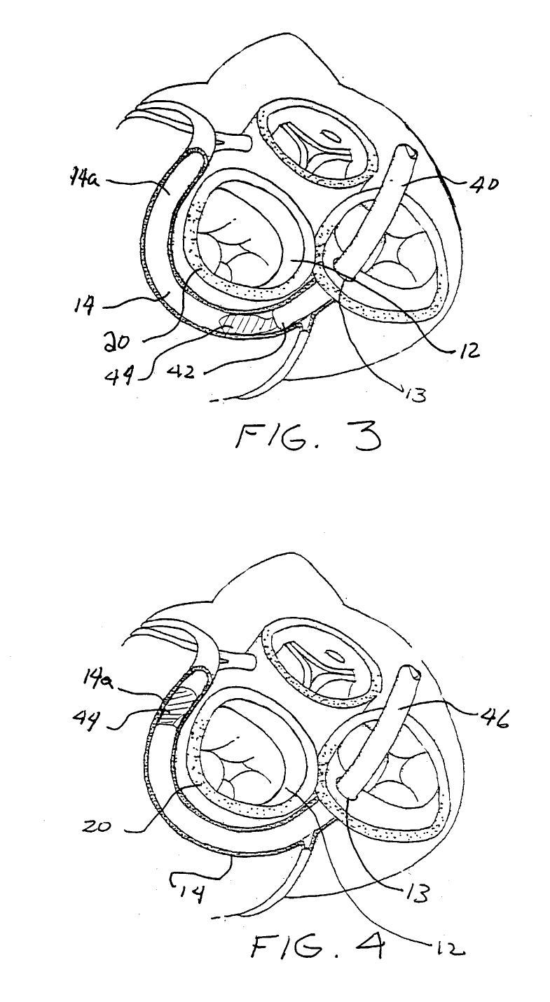

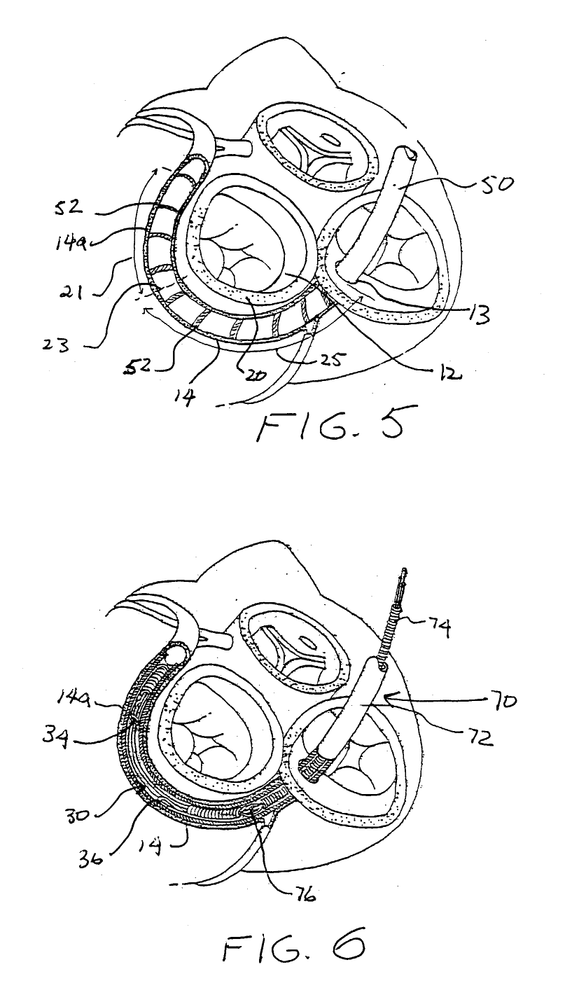

Next, the distal coronary sinus or great cardiac vein 14a is cannulated with a catheter 46 which again is inserted through the ostium 13 into the coronary sinus 14 and distally to the great cardiac vein 14a as shown in FIG. 4. A venogram is then performed on the great cardiac vein 14a by the injection of the contrast material 44. The venogram is performed in the great cardiac vein to assess the stretched and native diameter of the great cardiac vein at a point where the distal anchor 34 (FIG. 2) of the device 30 will be deployed.

Following the venogram of the great cardiac vein, a catheter 50 having marker bands 52 is deployed in the coronary sinus 14 and great cardiac vein 14a as illustrated in FIG. 5. The markers 52 are preferably visible under fluoroscopy and are spaced apart by a known distance. This enables the length 21 from the distal great vein to the great vein/coronary sinus junction 23 to be determined and the length 25 from the great vein/coronary sinus junction 23 to the ostium 13 to be determined.

At this point, the anatomy of the circumflex artery and its branches, the great vein, and the coronary sinus are recorded in terms of diameter, shape, and length. This enables the selection of a suitably dimensioned device for implant from a plurality of provided devices each having dimensions corresponding to a respective different set of anatomical features or dimensions. To complete the assessment of the device to be selected, the amount of mitral annulus reduction is estimated. This estimation is based upon the degree of mitral regurgitation, the coronary angiogram, and the venogram measurements. In most cases, a reduction in the mitral annular area will be on the order of 20%-60% as is illustrated, for example, with the deployed device 30 in FIG. 2.

The device 30 along with its deployment system 70 is illustrated in FIG. 6. As shown, the device is in the process of being implanted in the coronary sinus 14/great vein 14a of the heart 10. Its proximal anchor 36 and distal anchor 34 have not yet been deployed. The deployment system 70 includes an elongated catheter 72, an elongated pusher 74, and a coupling structure 76. The coupling structure is particularly shown and described in copending application Ser. No. 10/331,143, filed Dec. 26, 2002, titled SYSTEM AND METHOD TO EFFECT THE MITRAL VALVE ANNULUS OF A HEART, and which application is owned by the assignee of the present invention and incorporated herein by reference. As disclosed therein, the device 30 is releasably locked to the pusher 74 by the coupling structure 76.

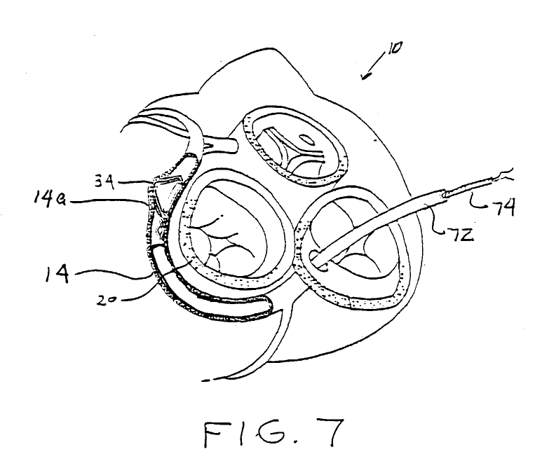

In deploying the device 30, the catheter 72 is first fed into the coronary sinus 14 adjacent the mitral valve annulus 20. The device 30 and pusher 54 at this time are releasably locked together. The device is then loaded into the catheter 72. The pusher 74 follows the device into the catheter 72 and is then advanced along the catheter to push the device 30 distally down the catheter to a predetermined position adjacent the mitral valve annulus 14 at the distal end of the catheter 72. Thereafter, the device is maintained in a stationary position by the pusher 74 as the catheter 72 is partially withdrawn to expose the distal anchor 34. The exposure of the distal anchor 34 may now be confirmed under fluoroscopy. It is then deployed in a manner as fully described in the aforementioned copending application Ser. No. 10/142,637. Once the distal anchor 34 is deployed, the pusher 74 is pulled proximally as shown in FIG. 7 for tightening the device within the coronary sinus and to an extent believed necessary to result in the desired effect on the geometry of the mitral valve annulus 20. During this adjustment process, mitral regurgitation may be monitored and the device tension adjusted to evaluate the effectiveness of the device for optimal results.

Once the device tension is adjusted for optimal results, arterial perfusion of the heart is assessed to determine if the tension on the device has adversely affected arterial perfusion of the heart. Heretofore, arterial perfusion has been assessed during or after procedures performed in the cardiac arterial system, such as after angioplasty or after implantation of a stent in a coronary artery. The assessment for arterial perfusion may be made in a number of different ways as known in the art. For example, the assessment may be made by performing one or more of the following: a coronary angiography, an intravascular ultrasound, a fractional flow reserve analysis, echocardiography, sampling for chemical markers of ischemia or myocardial ischemia detection via electrocardiogram.

Prior to this invention, however, the need to assess arterial perfusion during or after a procedure performed in the cardiac venous system (such as the mitral valve procedure described here) has not been recognized. By assessing both the efficacy of the procedure as well as the procedure's effect on cardiac perfusion, the clinician can maximize the benefit to the patient while minimizing potential harm to the patient. In this mitral valve procedure, therefore, the goal is to maximize arterial perfusion while minimizing mitral valve regurgitation. The desired amount of arterial perfusion and the tolerable amount of mitral valve regurgitation depend upon patient-dependent factors such as the patient's overall health, level of activity and extent of coronary artery disease.

Thus, prior to finalizing deployment of the device 30 in the coronary sinus, arterial perfusion of the patient's heart is assessed. For example, in performing the angiogram, the coronary arteries may be cannulated and injected with a contrast material viewable under fluoroscopy to define the anatomy and lumen diameter of the arterial system prior to deployment of the device in the coronary sinus or elsewhere in the cardiac venous system. After deployment, if the device crosses over a coronary artery and partially compresses the artery, the effect may be detected. While adequacy of arterial flow is a complex determination, the angiogram can help detect critical stenosis of key vessels.

If intravascular ultrasound is used to assess arterial perfusion, an intravascular ultrasound probe may be advanced into a coronary artery to determine the lumen diameter around the location of a device implanted in adjacent regions of the cardiac venous system, such as the coronary sinus. If the lumen of the artery is reduced by placement of the device, the intravascular ultrasound can quantitate the reduction.

As another example, in performing a fractional flow reserve analysis, a pressure wire is used to calculate the difference in pressures between the ascending aorta and the coronary artery. This enables one to detect whether or not significant stenosis exists within a coronary artery or vessel. After administering adenosine to the patient, placing a distal pressure transducer so that it is distal to the device in the coronary sinus would provide feedback regarding whether the placement of the device created significant arterial stenosis. For example, a ratio of distal to proximal pressure less than 0.7 may indicate an unacceptable reduction in arterial perfusion which would lead the clinician to adjust the implanted device.

With respect to echocardiography, when the myocardium experiences ischemia, it has the tendency to compromise contractility. Real-time echocardiography (transthoracic, transesophageal, and intracardiac) may be used as an indirect tool to determine if arterial blood supply is compromised to a sufficient degree to create myocardial ischemia. Dyskinesis, akinesis, hypokinesis, or dyssynchrony are all potential indicators of myocardial ischemia.

Another technique for monitoring arterial perfusion of the heart is to look for chemical markers of ischemia, such as troponin, creatine kinase and other techniques. Yet another technique is to use a doppler flow wire to monitor arterial flow rates.

Lastly, with respect to the detection for myocardial ischemia, an electrocardiogram may be taken from which ST segment changes may be detected. Preferably, the electrocardiogram is a 12-lead electrocardiogram which may also help to localize where the ischemia even occurs. To the extent that a device in the coronary sinus could affect perfusion in the anterior, posterior and lateral segments of a heart, an electrocardiogram could provide indirect evidence of myocardial ischemia.

Once adequate arterial perfusion is confirmed, deployment of the device 30 may be completed. This entails the retraction of the catheter 72 to expose the proximal anchor 36. The proximal anchor 36 may then be deployed as fully described in copending U.S. application Ser. No. 10/142,637. Once the device 30 is fully deployed, the coupling mechanism 76 releases the pusher 74 from the device 30. The pusher 74 and catheter 72 are then retracted from the patient.

With the device 30 now positioned in the heart as illustrated in FIG. 2, the effectiveness of the device may once again be confirmed. Also, it is preferable that another assessment of arterial perfusion be performed at this time to assure that perfusion of the heart has not been compromised.

If, after the device is deployed, additional adjustment is required, the deployment catheter 72 may be advanced into the coronary sinus partially over the proximal anchor to partially recapture it. Then, as fully described in the aforementioned copending application Ser. No. 10/331,143, tension may be imparted on the device for adjusting the device to the anatomy of the heart. If at any point during the procedure it is necessary to recapture one or both of the anchors to reposition or remove the device, the device may be recaptured as fully described in the aforementioned application Ser. No. 10/331,143. Once adequate arterial perfusion and mitral regurgitation reduction or elimination has been confirmed, the coupling structure 76 may uncouple the device from the pusher 74. This permits the deployment system 70 to be withdrawn from the patient.

While particular embodiments of the present invention have been shown and described, modifications may be made, and it is therefore intended in the appended claims to cover all such changes and modifications which fall within the true spirit and scope of the invention.

What is claimed:

1. A method of implanting a mitral valve therapy device in a patient's coronary sinus adjacent the patient's mitral valve annulus, the method comprising the steps of:
positioning the mitral valve therapy device within the coronary sinus of the patient adjacent to the mitral valve annulus of the patient;
evaluating effectiveness of the device; and
assessing arterial perfusion of the heart.

2. The method of claim 1 including the further step of adjusting the position of the device after the assessing step.

3. The method of claim 1 including the further step of removing the device after the assessing step.

4. The method of claim 1 wherein the device includes a distal anchor and a proximal anchor, wherein the positioning step includes deploying the distal anchor within the coronary sinus and wherein the evaluating step includes pulling proximally on the device.

5. The method of claim 4 wherein the assessing step is performed as the device is pulled proximally.

6. The method of claim 5 including the further step of deploying the proximal anchor while pulling proximally on the device.

7. The method of claim 6 including the further step of confirming effectiveness of the device after deploying the proximal anchor.

8. The method of claim 6 including the further step of assessing arterial perfusion of the heart after deploying the proximal anchor.

9. The method of claim 8 including the further step of recapturing the proximal anchor after the deploying and assessing steps.

10. The method of claim 8 including the further step of removing the device after the deploying and assessing steps.

11. The method of claim 1 wherein the assessing step includes performing coronary angiography.

12. The method of claim 1 wherein the assessing step includes intravascular ultrasound.

13. The method of claim 1 wherein the assessing step includes fractional flow reserve analysis.

14. The method of claim 1 wherein the assessing step includes performing an echocardiography.

15. The method of claim 1 wherein the assessing step includes the step of detecting for myocardial ischemia.

16. The method of claim 1 wherein the assessing step includes the step of detecting a chemical marker of ischemia.

17. The method of claim 15 wherein the detecting step includes taking an electrocardiogram.

18. The method of claim 1 including the further step of determining anatomical features of the coronary sinus adjacent to the mitral valve annulus.

19. The method of claim 18 wherein the determined anatomical features include one of shape, diameter, and length.

20. The method of claim 18 including the further steps of providing a plurality of mitral valve therapy devices, each device corresponding to a respective different set of the anatomical features and selecting one of the plurality of mitral valve therapy devices after determining the anatomical features of the coronary sinus.

21. A method of implanting a mitral valve therapy device in a patient's coronary sinus adjacent the patient's mitral valve annulus, the device including a distal anchor and a proximal anchor, the method comprising the steps of:
positioning the mitral valve therapy device within the coronary sinus adjacent to the mitral valve annulus;
deploying the distal anchor;
evaluating effectiveness of the device;
performing an arterial perfusion assessment of the heart;
deploying the proximal anchor; and
performing a second arterial perfusion assessment of the heart.

22. The method of claim 21 wherein the evaluating step includes pulling proximally on the device.

23. The method of claim 22 wherein the assessing step is performed as the device is pulled proximally.

24. The method of claim 23 wherein the proximal anchor is deployed while the device is pulled proximally.

25. The method of claim 21 including the further step of confirming effectiveness of the device after deploying the proximal anchor.

26. The method of claim 21 wherein at least one of the performing steps includes one of coronary angiography, intravascular ultrasound, fractional flow reserve analysis, echocardiography, and myocardial ischemia detection.

27. The method of claim 26 wherein the myocardial ischemia detection includes taking an electrocardiogram.

28. The method of claim 21 including the further step of determining anatomical features of the coronary sinus adjacent to the mitral valve annulus.

29. The method of claim 28 wherein the determined anatomical features include one of shape, diameter, and length.

30. The method of claim 28 including the further steps of providing a plurality of mitral valve therapy devices, each device corresponding to a respective different set of the anatomical features and selecting one of the plurality of mitral valve therapy devices after determining the anatomical features of the coronary sinus.

31. The method of claim 21 including the further step of recapturing the distal anchor after the step of performing an arterial perfusion assessment of the heart.

32. The method of claim 21 including the further step of recapturing the proximal anchor after the step of performing a second arterial perfusion assessment of the heart.

33. A method of optimizing patient outcome while performing a procedure in the venous system of a patient's heart, the method comprising the steps of:
performing a procedure in the venous system of the patient's heart;
evaluating effectiveness of the procedure; and assessing arterial perfusion of the heart.

34. The method of claim 33 including the further step of performing a further procedure in the venous system of the patient's heart after the evaluating step.

35. The method of claim 33 wherein the performing step comprises positioning a mitral valve therapy device within the coronary sinus adjacent to the mitral valve annulus of the patient's heart.

36. The method of claim 35 including the further step of repositioning the device after the evaluating step.

37. The method of claim 35 including the further step of removing the device after the evaluating step.
